(19) United States
(12) Patent Application Publication
(10) Pub. No.: US 2024/0384345 A1
(43) Pub. Date: Nov. 21, 2024

(54) METHOD FOR ASSESSING RISK OF CHRONIC KIDNEY DISEASE AND CHRONIC KIDNEY DISEASE RISK ASSESSMENT SYSTEM

(71) Applicant: China Medical University, Taichung City (TW)

(72) Inventors: Chin-Chi Kuo, Taichung City (TW); Hung-Lin Chen, Taichung City (TW); Hsiu-Yin Chiang, Taichung City (TW)

(73) Assignee: China Medical University, Taichung City (TW)

(21) Appl. No.: 18/533,936

(22) Filed: Dec. 8, 2023

Related U.S. Application Data
(60) Provisional application No. 63/466,379, filed on May 15, 2023.

(51) Int. Cl. C12Q 1/6883; G16B 5/00; G16B 20/20; G16B 30/10; G16B 40/20; G16H 50/30; G16H 50/70

(52) U.S. Cl. CPC C12Q 1/6883; G16B 5/00; G16B 20/20; G16B 30/10; G16B 40/20; G16H 50/30; G16H 50/70; C12Q 2600/118; C12Q 2600/156

(57) ABSTRACT

A method for assessing risk of chronic kidney disease includes following steps. A reference database is provided. A nucleic acid sample and a biological dataset of a subject are provided. A genetic testing step is performed. A risk score calculating step is performed. A model establishing step is performed, wherein a plurality of reference polygenic risk score data, a plurality of reference clinical data and a plurality of reference genetic marker data of the reference database are trained to achieve a convergence by a machine learning algorithm so as to obtain an analysis model. A data analysis step is performed, wherein a polygenic risk score, a clinical data and a genetic marker data of a subject are analyzed by the analysis model so as to obtain a risk analysis result.

### RELATED APPLICATIONS

This application claims priority to U.S. Provisional Application Ser. No. 63/466,379, filed May 15, 2023, which is herein incorporated by reference.

### BACKGROUND

### Technical Field

The present disclosure relates to a medical information analysis method and a system thereof. More particularly, the present disclosure relates to a method for assessing risk of chronic kidney disease and a chronic kidney disease risk assessment system.

### Description of Related Art

Chronic kidney disease (CKD) is a prevalent chronic disease worldwide. Owing to the lack of early prediction and definitive treatment method, the prevalence of chronic kidney disease and end-stage renal disease continues to increase, and the chronic kidney disease and the end-stage renal disease have become a global public health issue. According to the statistical results from the World Health Organization and the published literature, approximately 5% to 10% of the population worldwide suffers from the chronic kidney disease, and the global all-age prevalence of the chronic kidney disease increased by 29.3% from 1990 to 2017.

The treatment and management of chronic kidney disease require enormous medical resources and financial expenditures, and it places a heavy burden on individuals and society. Therefore, establishing a method that can early detect and assess the risk of chronic kidney disease is favorable for preventing and treating the chronic kidney disease early, thereby reducing the burden on individuals and society.

Currently, risk prediction models for the chronic kidney disease are mainly based on the assessment of clinical factors, such as an age, a sex, an estimated glomerular filtration rate (eGFR) and concentrations of albumin, calcium, phosphate and bicarbonate so as to predict the risk of a patient developing to the end-stage renal disease in 2 to 5 years. However, in recent years, studies have found that the chronic kidney disease is related to genetic factors, and the genetic background of different populations influences the association between genes and diseases. Therefore, a method is needed to predict the risk of chronic kidney disease for individuals with different genetic backgrounds.

### SUMMARY

According to one aspect of the present disclosure, a method for assessing risk of chronic kidney disease includes following steps. A reference database is provided, wherein the reference database includes a plurality of target gene data and a reference ontological dataset, and the reference ontological dataset includes a plurality of reference polygenic risk score data, a plurality of reference clinical data and a plurality of reference genetic marker data. A nucleic acid sample and a biological dataset of a subject are provided, wherein the biological dataset includes a clinical data and at least one genetic marker data. A genetic testing step is performed, wherein a plurality of single nucleotide polymorphisms (SNPs) of the nucleic acid sample are simultaneously detected by a nucleic acid detection method and are compared to the plurality of target gene data so as to obtain a genotyping result. A risk score calculating step is performed, wherein the genotyping result is calculated by a polygenic risk score assessing method so as to obtain a polygenic risk score. A model establishing step is performed, wherein the plurality of reference polygenic risk score data, the plurality of reference clinical data and the plurality of reference genetic marker data are trained to achieve a convergence by a machine learning algorithm so as to obtain an analysis model. A data analysis step is performed, wherein the polygenic risk score, the clinical data and the at least one genetic marker data are analyzed by the analysis model so as to obtain a risk analysis result, and the risk analysis result is for assessing an age at which the subject is suffered from a chronic kidney disease.

According to another aspect of the present disclosure, a chronic kidney disease risk assessment system includes a non-transitory machine readable medium, a genetic testing device and a processor. The non-transitory machine readable medium is for storing a biological dataset of a subject and a reference database, wherein the biological dataset includes a nucleic acid sample, a clinical data and at least one genetic marker data, the reference database includes a plurality of target gene data and a reference ontological dataset, and the reference ontological dataset includes a plurality of reference polygenic risk score data, a plurality of reference clinical data and a plurality of reference genetic marker data. The genetic testing device is signally connected to the non-transitory machine readable medium, wherein the genetic testing device is for detecting and comparing a plurality of single nucleotide polymorphisms of the nucleic acid sample to the plurality of target gene data so as to obtain a genotyping result. The processor is signally connected to the genetic testing device and the non-transitory machine readable medium, wherein the processor includes a polygenic risk score software and a risk assessment program, and the genotyping result is calculated by the polygenic risk score software so as to obtain a polygenic risk score. The risk assessment program includes an analysis model, and the polygenic risk score, the clinical data and the at least one genetic marker data are analyzed by the analysis model so as to obtain a risk analysis result, and the risk analysis result is for assessing an age at which the subject is suffered from a chronic kidney disease.

### BRIEF DESCRIPTION OF THE DRAWINGS

The present disclosure can be more fully understood by reading the following detailed description of the embodiment, with reference made to the accompanying drawings as follows:

FIG. 1 is a flow chart of a method for assessing risk of chronic kidney disease according to the first embodiment of the present disclosure.

FIG. 2 is a flow chart of a method for assessing risk of chronic kidney disease according to the second embodiment of the present disclosure.

FIG. 3 is a block diagram of a chronic kidney disease risk assessment system according to the third embodiment of the present disclosure.

FIG. 4 shows a risk analysis result obtained from a chronic kidney disease risk assessment system of the present disclosure.

FIG. 5 shows a risk analysis result of the chronic kidney disease risk assessment system of the present disclosure for the Taiwanese population.

FIG. 6 shows a risk analysis result of the chronic kidney disease risk assessment system of the present disclosure for the U.K. population.

FIG. 7 shows discriminant statistical results of the analysis model of the present disclosure for the Taiwanese population and the U.K. population in the testing dataset.

FIG. 8 shows a forest plot comparison of the odds ratio (OR) of risk of chronic kidney disease by groups with different polygenic risk scores.

### DETAILED DESCRIPTION

The present disclosure will be further exemplified by the following specific embodiments to facilitate utilizing and practicing the present disclosure completely by the people skilled in the art without over-interpreting and over-experimenting. However, these practical details are used to describe how to implement the materials and methods of the present disclosure and are not necessary.

### Method for Assessing Risk of Chronic Kidney Disease of the Present Disclosure

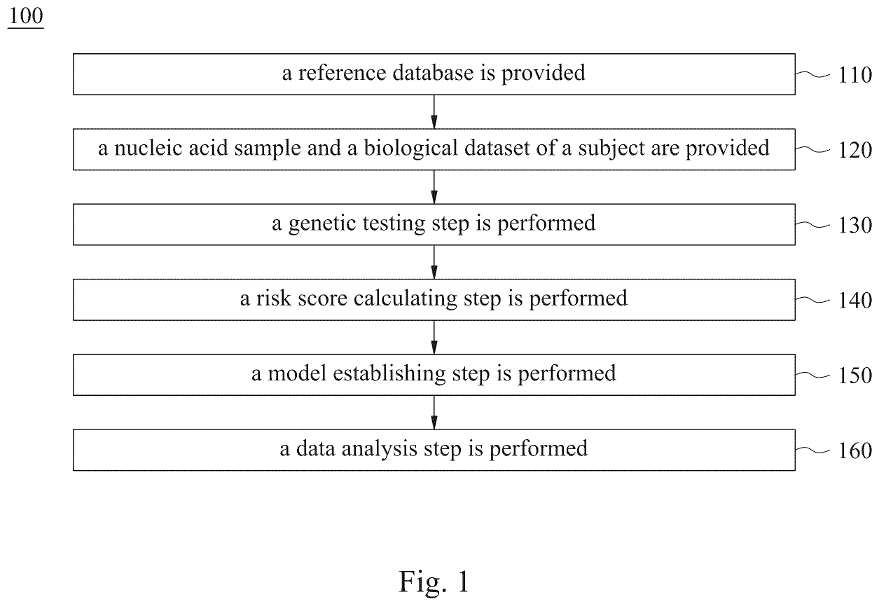

Reference is made to FIG. 1, which is a flow chart of a method 100 for assessing risk of chronic kidney disease according to the first embodiment of the present disclosure. The method 100 for assessing risk of chronic kidney disease includes Step 110, Step 120, Step 130, Step 140, Step 150 and Step 160.

In Step 110, a reference database is provided, wherein the reference database includes a plurality of target gene data and a reference ontological dataset, and the reference ontological dataset includes a plurality of reference polygenic risk score data, a plurality of reference clinical data and a plurality of reference genetic marker data.

In particular, the plurality of target gene data include a plurality of gene data related to a chronic kidney disease, wherein the plurality of target gene data can include ASCC3 gene data, CDC1 gene data, F12 gene data, FAM47E gene data, FBXO22 gene data, HCRTR2 gene data, KNG1 gene data, LRP2 gene data, RAI14 gene data, NRG4 gene data, PAX8 gene data, PDILT gene data, SIM1 gene data, STC1 gene data, TINAG gene data, UBE2Q2 gene data and WDR72 gene data, and the present disclosure is not limited thereto.

The reference ontological dataset can be a dataset obtained from the results of patient diagnosis, laboratory tests and data analysis in electronic medical records (EMRs) of subjects in a hospital, and the plurality of reference polygenic risk score data, the plurality of reference clinical data and the plurality of reference genetic marker data used in the present disclosure are further described as follows.

In particular, the plurality of reference polygenic risk score data are obtained from the results of genotype imputation in a plurality of gene data of the subjects, wherein one of the plurality of gene data of each of the subjects is obtained from the result of genetic testing, and each of the plurality of reference polygenic risk score data is a comprehensive score calculated from genotypes of multiple genetic loci of each of the subjects and the genotypes of multiple genetic loci corresponding weights.

The plurality of reference clinical data include a plurality of reference age data, a plurality of reference sex data, a plurality of reference family medical history data, a plurality of reference personal medical history data, a plurality of reference medication history data and a plurality of reference biochemical testing data of the subjects, wherein each of the plurality of reference biochemical testing data can be a blood test data including a reference estimated glomerular filtration rate (eGFR) data, a reference albumin concentration data, a reference calcium concentration data, a reference phosphate concentration data and a reference bicarbonate concentration data.

The plurality of reference genetic marker data can include a plurality of molecular genetic marker data, wherein the plurality of molecular genetic marker data can include a plurality of reference RNA level data and a plurality of reference protein level data of the subjects, and the plurality of reference RNA level data and the plurality of reference protein level data can correspond to the RNA levels and the protein levels of the plurality of target gene data. Therefore, it is favorable for enhancing the assessment accuracy of an analysis model trained with the plurality of reference genetic marker data.

In Step 120, a nucleic acid sample and a biological dataset of a subject are provided, wherein the biological dataset includes a clinical data and at least one genetic marker data. In particular, the subject can be an inpatient or an outpatient, the nucleic acid sample can be a blood, a urine, a saliva, or a combination thereof, and the present disclosure is not limited thereto. Further, the clinical data can include an age data, a sex data, a family medical history data, a personal medical history data, a medication history data and a biochemical testing data, wherein the biochemical testing data can include an estimated glomerular filtration rate data, an albumin concentration data, a calcium concentration data, a phosphate concentration data and a bicarbonate concentration data. Furthermore, the clinical data can further include a lifestyle data, wherein the lifestyle data includes a food frequency data, a smoking habit data and an alcohol usage data, and the present disclosure is not limited thereto. Moreover, the genetic marker data can include a plurality of RNA level data and a plurality of protein level data, wherein the plurality of RNA level data and the plurality of protein level data can correspond to the RNA levels and the protein levels of the plurality of target gene data.

In Step 130, a genetic testing step is performed, wherein a plurality of single nucleotide polymorphisms (SNPs) of the nucleic acid sample are simultaneously detected by a nucleic acid detection method and are compared to the plurality of target gene data so as to obtain a genotyping result. In detail, the nucleic acid detection method can be a polymerase chain reaction (PCR) method, a gene chip analyzing method or a next generation sequencing (NGS) method, and the present disclosure is not limited thereto.

In Step 140, a risk score calculating step is performed, wherein the genotyping result is calculated by a polygenic risk score assessing method so as to obtain a polygenic risk score. In particular, the polygenic risk score assessing method can be performed by a PRSice-2 software, and the polygenic risk score is calculated by the PRSice-2 software based on the following formula:

$$P R {S}_{j} = {\sum }_{i}^{N} {β}_{i} {dosage}_{i , j} ,$$

wherein “PRS” represents the polygenic risk score, “N” represents a number of the single nucleotide polymorphisms in the score, “βᵢ” represents an effect size of variant “i”, and “dosagei,j” represents a number of copies of single nucleotide polymorphisms in the genotype of individual “j”.

Further, the calculation details of the polygenic risk score are well-known in the art, so the detailed steps thereof will not be described herein again.

In Step 150, a model establishing step is performed, wherein the plurality of reference polygenic risk score data, the plurality of reference clinical data and the plurality of reference genetic marker data are trained to achieve a convergence by a machine learning algorithm so as to obtain an analysis model. Particularly, in the method 100 for assessing risk of chronic kidney disease, the machine learning algorithm can be a linear regression algorithm, a logistic regression algorithm, a decision tree algorithm or a random forest algorithm, and the present disclosure is not limited thereto.

In Step 160, a data analysis step is performed, wherein the polygenic risk score, the clinical data and the at least one genetic marker data are analyzed by the analysis model so as to obtain a risk analysis result, and the risk analysis result is for assessing an age at which the subject is suffered from a chronic kidney disease. In detail, the risk analysis result includes cumulative incidence curves for the chronic kidney disease across polygenic risk categories, wherein the polygenic risk categories can be grouped based on the ranking result of the polygenic risk scores, and the cumulative incidence curves can be used to assess the age at which the subject is suffered from chronic kidney disease.

Therefore, by analyzing the polygenic risk score, the clinical data and the genetic marker data of the subject by the analysis model, and the analysis model is established by training the plurality of reference polygenic risk score data, the plurality of reference clinical data and the plurality of reference genetic marker data of the reference ontological dataset, the method 100 for assessing risk of chronic kidney disease of the present disclosure can rapidly and accurately output the risk analysis result of the subject. Hence, it is favorable for the diagnosis of the chronic kidney disease and designing the subsequent medical plans of the subject, and thus the method 100 for assessing risk of chronic kidney disease of the present disclosure has excellent clinical application potential.

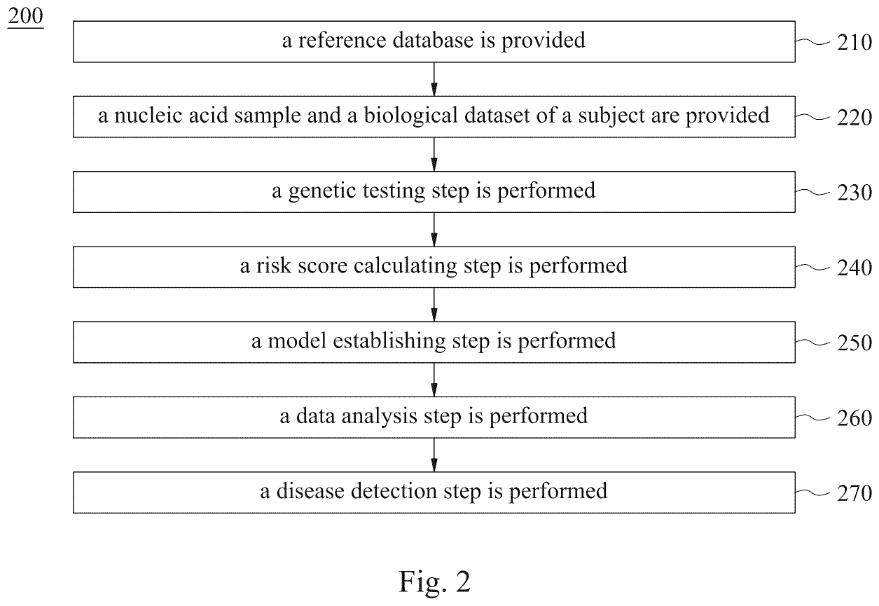

Reference is made to FIG. 2, which is a flow chart of a method 200 for assessing risk of chronic kidney disease according to the second embodiment of the present disclosure. The method 200 for assessing risk of chronic kidney disease includes Step 210, Step 220, Step 230, Step 240, Step 250, Step 260 and Step 270, wherein Step 210, Step 220, Step 230, Step 240, Step 250 and Step 260 are the same with Step 110, Step 120, Step 130, Step 140, Step 150 and Step 160 of FIG. 1, so that the same details there between are not described again herein.

In Step 270, a disease detection step is performed. In detail, according to the risk analysis result obtained from the analysis model, regular follow-up examinations should be conducted on the subject to confirm whether a biochemical indicator of the subject is abnormal, and thus it is favorable for checking whether the subject is suffered from an early-stage chronic kidney disease or not. Further, the biochemical indicator can include a level of urine protein, a blood pressure, a concentration of creatinine, a concentration of calcium and a concentration of potassium, and the present disclosure is not limited thereto.

Therefore, by that the biochemical indicator of the subject is regularly examined, the chronic kidney disease can be treated and managed earlier when the symptoms of the chronic kidney disease are detected early, and the treatment effect and quality of life of the subject can be improved. Hence, the method 200 for assessing risk of chronic kidney disease has excellent clinical application potential.

### Chronic Kidney Disease Risk Assessment System of the Present Disclosure

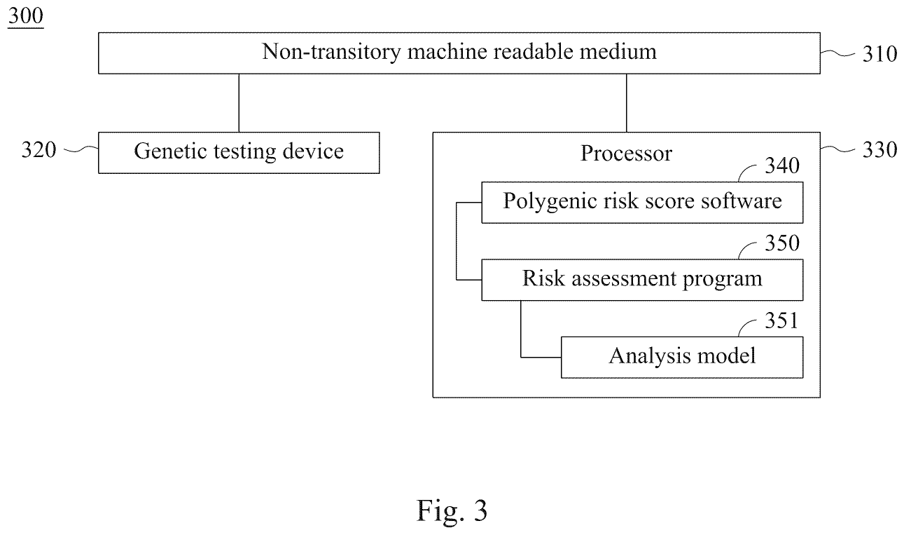

Reference is made to FIG. 3, which is a block diagram of a chronic kidney disease risk assessment system 300 according to the third embodiment of the present disclosure. The chronic kidney disease risk assessment system 300 includes a non-transitory machine readable medium 310, a genetic testing device 320 and a processor 330.

The non-transitory machine readable medium 310 is for storing a biological dataset of a subject and a reference database, wherein the biological dataset includes a nucleic acid sample, a clinical data and at least one genetic marker data, the reference database includes a plurality of target gene data and a reference ontological dataset, and the reference ontological dataset includes a plurality of reference polygenic risk score data, a plurality of reference clinical data and a plurality of reference genetic marker data.

In detail, the nucleic acid sample can be a blood, a urine, a saliva, or a combination thereof. The clinical data can include an age data, a sex data, a family medical history data, a personal medical history data, a medication history data and a biochemical testing data, wherein the biochemical testing data can include an estimated glomerular filtration rate data, an albumin concentration data, a calcium concentration data, a phosphate concentration data and a bicarbonate concentration data. Furthermore, the clinical data can further include a lifestyle data, wherein the lifestyle data includes a food frequency data, a smoking habit data and an alcohol usage data, and the present disclosure is not limited thereto. Moreover, the genetic marker data can include a plurality of RNA level data and a plurality of protein level data, wherein the plurality of RNA level data and the plurality of protein level data can correspond to the RNA levels and the protein levels of the plurality of target gene data.

The plurality of target gene data can include ASCC3 gene data, CDC1 gene data, F12 gene data, FAM47 E gene data, FBXO22 gene data, HCRTR2 gene data, KNG1 gene data, LRP2 gene data, RAI14 gene data, NRG4 gene data, PAX8 gene data, PDILT gene data, SIM1 gene data, STC1 gene data, TINAG gene data, UBE2 Q2 gene data and WDR72 gene data, and the present disclosure is not limited thereto.

The reference ontological dataset can be a dataset obtained from the results of patient diagnosis, laboratory tests and data analysis in electronic medical records of subjects in a hospital, wherein the plurality of reference polygenic risk score data are obtained from the results of genotype imputation in a plurality of gene data of the subjects, one of the plurality of gene data of each of the subjects is obtained from the result of genetic testing, and each of the plurality of reference polygenic risk score data is a comprehensive score calculated from genotypes of multiple genetic loci of each of the subjects and the genotypes of multiple genetic loci corresponding weights.

The plurality of reference clinical data include a plurality of reference age data, a plurality of reference sex data, a plurality of reference family medical history data, a plurality of reference personal medical history data, a plurality of reference medication history data and a plurality of reference biochemical testing data of the subjects, wherein each of the plurality of reference biochemical testing data can be a blood test data including a reference estimated glomerular filtration rate data, a reference albumin concentration data, a reference calcium concentration data, a reference phosphate concentration data and a reference bicarbonate concentration data.

The plurality of reference genetic marker data can include a plurality of molecular genetic marker data, wherein the plurality of molecular genetic marker data can include a plurality of reference RNA level data and a plurality of reference protein level data of the subjects, and the plurality of reference RNA level data and the plurality of reference protein level data can correspond to the RNA levels and the protein levels of the plurality of target gene data. Therefore, it is favorable for enhancing the assessment accuracy of an analysis model trained with the plurality of reference genetic marker data.

The genetic testing device 320 is signally connected to the non-transitory machine readable medium 310, wherein the genetic testing device 320 is for detecting and comparing a plurality of single nucleotide polymorphisms of the nucleic acid sample to the plurality of target gene data so as to obtain a genotyping result. In detail, the plurality of single nucleotide polymorphisms can be simultaneously detected by a polymerase chain reaction method, a gene chip analyzing method or a next generation sequencing method.

The processor 330 is signally connected to the genetic testing device 320 and the non-transitory machine readable medium 310, wherein the processor 330 includes a polygenic risk score software 340 and a risk assessment program 350, and the genotyping result is calculated by the polygenic risk score software 340 so as to obtain a polygenic risk score. In detail, the polygenic risk score software 340 can be a PRSice-2 software. Further, the calculation details of the polygenic risk score are described in the aforementioned paragraphs, so the detailed steps thereof will not be described herein again.

The risk assessment program 350 includes an analysis model 351, and the polygenic risk score, the clinical data and the at least one genetic marker data are analyzed by the analysis model 351 so as to obtain a risk analysis result, and the risk analysis result is for assessing an age at which the subject is suffered from a chronic kidney disease. Particularly, the analysis model can be obtained by training the plurality of reference polygenic risk score data, the plurality of reference clinical data and the plurality of reference genetic marker data to achieve a convergence by a machine learning algorithm, wherein the machine learning algorithm can be a linear regression algorithm, a logistic regression algorithm, a decision tree algorithm or a random forest algorithm.

Therefore, by analyzing the polygenic risk score, the clinical data and the genetic marker data of the subject by the analysis model 351 of the present disclosure, the chronic kidney disease risk assessment system 300 of the present disclosure can rapidly and accurately output the risk analysis result of the subject for designing the subsequent medical plans of the subject, and thus the chronic kidney disease risk assessment system 300 of the present disclosure has excellent clinical application potential.

### Example

### I. Reference Database

The reference database used in the present disclosure is collected by China Medical University Hospital and obtained from the electronic medical records of the subjects, wherein the subjects include healthy individuals and chronic kidney disease patients.

The following examples will be performed by the chronic kidney disease risk assessment system of the present disclosure supplemented by the method for assessing risk of chronic kidney disease of the present disclosure so as to evaluate the assessment accuracy of the chronic kidney disease risk assessment system and the method for assessing risk of chronic kidney disease of the present disclosure. The chronic kidney disease risk assessment system of the present disclosure can be the chronic kidney disease risk assessment system 300 described as the foregoing statement, and the method for assessing risk of chronic kidney disease of the present disclosure can be the method 100 for assessing risk of chronic kidney disease or the method 200 for assessing risk of chronic kidney disease, so that the details thereof are shown in the aforementioned paragraphs and not be described again herein.

### II. Results

### 1. Risk Analysis Result of the Present Disclosure

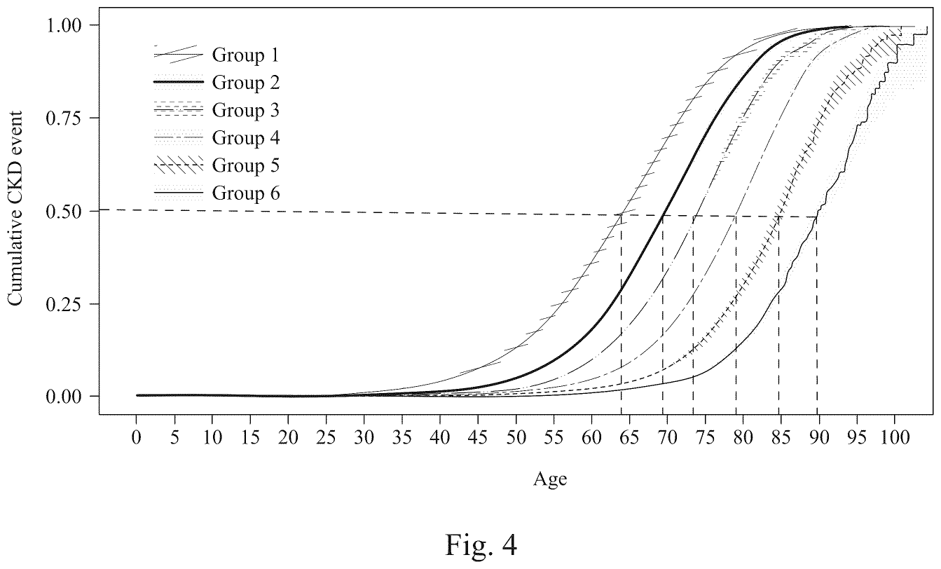

Reference is made to FIG. 4 and Table 1. FIG. 4 shows a risk analysis result obtained from the chronic kidney disease risk assessment system of the present disclosure. Table 1 shows estimated ages at which subjects classified in different groups are suffered from chronic kidney disease in FIG. 4.

TABLE 1

| | Polygenic risk category | Estimated age |
|---|---|---|
| Group | (of the polygenic risk scores) | (years old) |
| 1 | Top 2% | 64.8 |
| 2 | Top 2%-10% | 69.8 |
| 3 | Top 10%-20% | 73.9 |
| 4 | Median 20%-60% | 79.3 |
| 5 | Bottom 20%-40% | 84.8 |
| 6 | Bottom 20% | 89.9 |

As shown in FIG. 4, the risk analysis result obtained from the chronic kidney disease risk assessment system includes cumulative incidence curves for the chronic kidney disease (CKD) across polygenic risk categories, wherein the polygenic risk categories are grouped based on the ranking result of the polygenic risk scores, and the polygenic risk categories are divided into six groups in FIG. 4. The six groups of polygenic risk categories are respectively the top 2% of the polygenic risk scores (Group 1), the top 2%-10% of the polygenic risk scores (Group 2), the top 10%-20% of the polygenic risk scores (Group 3), the median 20%-60% of the polygenic risk scores (Group 4), the bottom 20%-40% of the polygenic risk scores (Group 5) and the bottom 20% of the polygenic risk scores (Group 6), and the p-value of the log-rank test is less than 0.0001.

As shown in FIG. 4 and Table 1, the subjects classified in Group 1 are likely to develop chronic kidney disease at age 64.8 years old, and the subjects classified in Group 6 are likely to develop chronic kidney disease at age 89.9 years old. Hence, the risk analysis result obtained from the analysis model of the present disclosure can be used to assess the age at which the subject is suffered from chronic kidney disease, thereby realizing ultra-early-stage risk prediction of the chronic kidney disease.

### 2. Performance of the Analysis Model of the Present Disclosure in Assessing the Risk of Chronic Kidney Disease in the Taiwanese Population and the United Kingdom (“U.K.” Hereafter) Population

In order to evaluate the performance of the analysis model of the chronic kidney disease risk assessment system of the present disclosure, an internal testing dataset and an external testing dataset are used to confirm the accuracy of the analysis model of the chronic kidney disease risk assessment system of the present disclosure in the Asian population and the European population. In detail, the internal testing dataset is collected from Taiwan Biobank, and the external testing dataset is collected from U.K. Biobank, wherein the Asian population is analyzed by using the biological dataset of the Taiwanese population as a representative, and the European population is analyzed by using the biological dataset of the U.K. population as a representative.

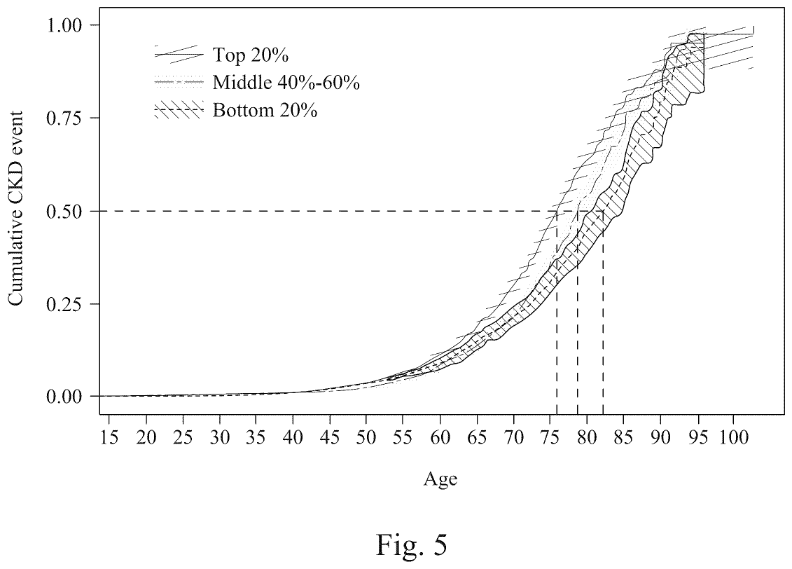

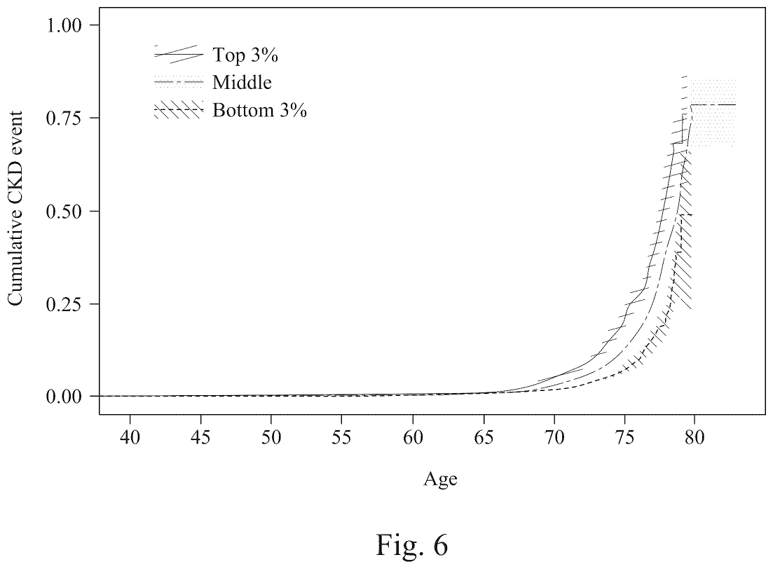

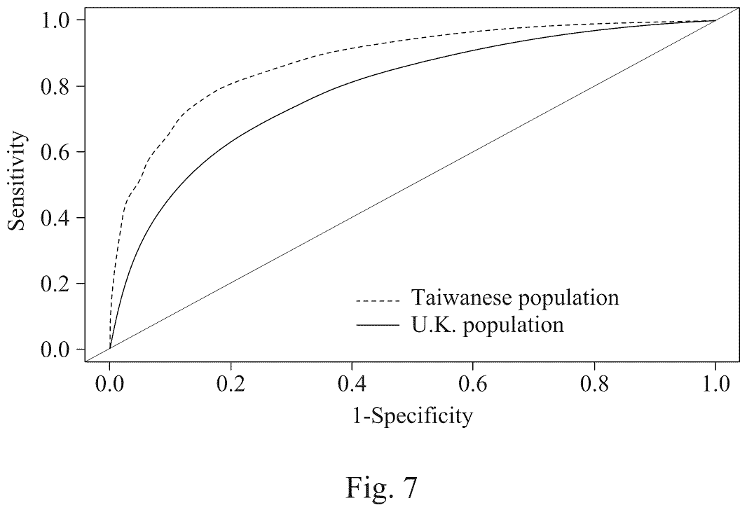

Reference is made to FIG. 5 to FIG. 7. FIG. 5 shows a risk analysis result of the chronic kidney disease risk assessment system of the present disclosure for the Taiwanese population. FIG. 6 shows a risk analysis result of the chronic kidney disease risk assessment system of the present disclosure for the U.K. population. FIG. 7 shows discriminant statistical results of the analysis model of the present disclosure for the Taiwanese population and the U.K. population in the testing dataset. Particularly, in FIG. 7, the area under the receiver operating characteristic curve (“AUC” hereafter) of the Taiwanese population is 0.880 (0.875-0.885), and the AUC of the U.K. population is 0.788 (0.783-0.793).

As shown in FIG. 5 and FIG. 6, the risk analysis results obtained from the chronic kidney disease risk assessment system include cumulative incidence curves for the chronic kidney disease across polygenic risk categories, wherein the polygenic risk categories are grouped based on the ranking result of the polygenic risk scores. In FIG. 5, the polygenic risk categories are divided into three groups. The three groups of polygenic risk categories in FIG. 5 are respectively the top 20% of the polygenic risk scores, the middle 40%-60% of the polygenic risk scores and the bottom 20% of the polygenic risk scores, wherein the p-value of the log-rank test is less than 0.0001. In FIG. 6, the polygenic risk categories are also divided into three groups. The three groups of polygenic risk categories in FIG. 6 are respectively the top 3% of the polygenic risk scores, the middle of the polygenic risk scores and the bottom 3% of the polygenic risk scores, and the p-value of the log-rank test is also less than 0.0001.

As further shown in FIG. 5 and FIG. 6, the chronic kidney disease risk assessment system and the method for assessing risk of chronic kidney disease of the present disclosure can be used to assess the age at which a subject is suffered from chronic kidney disease, wherein the subject can have an Asian genetic background or a European genetic background.

As shown in FIG. 7, the AUCs of the chronic kidney disease risk assessment system and the method for assessing risk of chronic kidney disease of the present disclosure are more than 0.7 when assessing the Taiwanese population and the U.K. population. In particular, the AUC equal to 0.5 indicates that a model has no class separation capacity. As the AUC continues to improve, the class separation capacity of the assessment model is getting better and better. Compared with the AUC of the U.K. population, the AUC of the Taiwanese population assessed by the chronic kidney disease risk assessment system and the method for assessing risk of chronic kidney disease of the present disclosure can reach 0.880, which shows the accuracy of the present disclosure for assessing chronic kidney disease in the Asian population.

### 3. The Effect of the Polygenic Risk Score in Odds Ratio (OR) of Chronic Kidney Disease

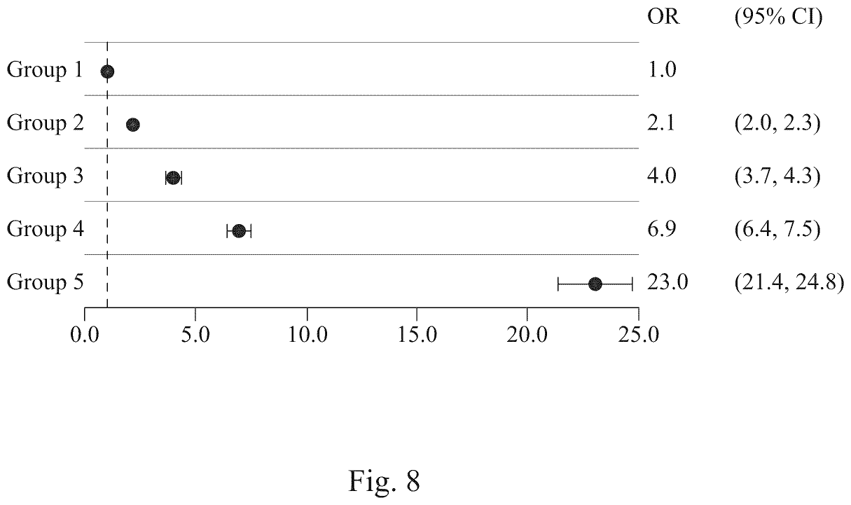

Reference is made to FIG. 8, which shows a forest plot comparison of the odds ratio of risk of chronic kidney disease by groups with different polygenic risk scores. In detail, the polygenic risk categories are divided into five groups, wherein the five groups of polygenic risk categories are respectively the bottom less than 20% of the polygenic risk scores (Group 1), the bottom 20%-39% of the polygenic risk scores (Group 2), the middle 41%-60% of the polygenic risk scores (Group 3), the top 21%-40% of the polygenic risk scores (Group 4) and the top 20% of the polygenic risk scores (Group 5), and Group 1 is used to a reference group. Further, “95% CI” represents a 95% confidence interval.

As shown in FIG. 8, the subjects classified in Group 5 have a 23-fold higher risk of developing chronic kidney disease than those classified in Group 1.

As shown in the aforementioned results, the method for assessing risk of chronic kidney disease and the chronic kidney disease risk assessment system of the present disclosure can be used to assess the age at which the subject may develop the chronic kidney disease, thereby realizing ultra-early-stage risk prediction of the chronic kidney disease. Further, the method for assessing risk of chronic kidney disease and the chronic kidney disease risk assessment system of the present disclosure can be used to assess the risk of chronic kidney disease in subjects with different genetic background, and the method for assessing risk of chronic kidney disease and the chronic kidney disease risk assessment system of the present disclosure have excellent assessment accuracy. Furthermore, the impact of the family medical history of chronic kidney disease on individual risk can be evaluated by the method for assessing risk of chronic kidney disease and the chronic kidney disease risk assessment system of the present disclosure so as to enhance the assessment accuracy of chronic kidney disease. Moreover, the present disclosure is developed based on the Asian genes with the maximum sample size, which has a wide range of sample collection and can provide an improved accurate risk assessment and prediction for patients with the chronic kidney disease in Asia, thereby improving the kidney function of the Asian population and reducing the burden of medical expenses. Therefore, it is favorable for the diagnosis of the chronic kidney disease and designing the subsequent medical plans of the subject, and thus the method for assessing risk of chronic kidney disease and the chronic kidney disease risk assessment system of the present disclosure have excellent assessment accuracy, and have important clinical application potential and commercial value.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A method for assessing risk of chronic kidney disease, comprising:
providing a reference database, wherein the reference database comprises a plurality of target gene data and a reference ontological dataset, and the reference ontological dataset comprises a plurality of reference polygenic risk score data, a plurality of reference clinical data and a plurality of reference genetic marker data;
providing a nucleic acid sample and a biological dataset of a subject, wherein the biological dataset comprises a clinical data and at least one genetic marker data;
performing a genetic testing step, wherein a plurality of single nucleotide polymorphisms (SNPs) of the nucleic acid sample are simultaneously detected by a nucleic acid detection method and are compared to the plurality of target gene data so as to obtain a genotyping result;
performing a risk score calculating step, wherein the genotyping result is calculated by a polygenic risk score assessing method so as to obtain a polygenic risk score;
performing a model establishing step, wherein the plurality of reference polygenic risk score data, the plurality of reference clinical data and the plurality of reference genetic marker data are trained to achieve a convergence by a machine learning algorithm so as to obtain an analysis model; and
performing a data analysis step, wherein the polygenic risk score, the clinical data and the at least one genetic marker data are analyzed by the analysis model so as to obtain a risk analysis result, and the risk analysis result is for assessing an age at which the subject is suffered from a chronic kidney disease.

2. The method of claim 1, wherein the plurality of target gene data comprise ASCC3 gene data, CDC1 gene data, F12 gene data, FAM47 E gene data, FBXO22 gene data, HCRTR2 gene data, KNG1 gene data, LRP2 gene data, RAI14 gene data, NRG4 gene data, PAX8 gene data, PDILT gene data, SIM1 gene data, STC1 gene data, TINAG gene data, UBE2 Q2 gene data and WDR72 gene data.

3. The method of claim 1, wherein the nucleic acid sample is a blood, a urine, a saliva, or a combination thereof.

4. The method of claim 1, wherein the nucleic acid detection method is a polymerase chain reaction (PCR) method, a gene chip analyzing method or a next generation sequencing (NGS) method.

5. The method of claim 1, wherein the polygenic risk score assessing method is performed by a PRSice-2 software.

6. The method of claim 1, wherein the machine learning algorithm is a linear regression algorithm, a logistic regression algorithm, a decision tree algorithm or a random forest algorithm.

7. The method of claim 1, wherein the clinical data comprises an age data, a sex data, a family medical history data, a personal medical history data, a medication history data and a biochemical testing data.

8. The method of claim 7, wherein the biochemical testing data comprises an estimated glomerular filtration rate (eGFR) data, an albumin concentration data, a calcium concentration data, a phosphate concentration data and a bicarbonate concentration data.

9. A chronic kidney disease risk assessment system, comprising:
a non-transitory machine readable medium for storing a biological dataset of a subject and a reference database, wherein the biological dataset comprises a nucleic acid sample, a clinical data and at least one genetic marker data, the reference database comprises a plurality of target gene data and a reference ontological dataset, and the reference ontological dataset comprises a plurality of reference polygenic risk score data, a plurality of reference clinical data and a plurality of reference genetic marker data;
a genetic testing device signally connected to the non-transitory machine readable medium, wherein the genetic testing device is for detecting and comparing a plurality of single nucleotide polymorphisms of the nucleic acid sample to the plurality of target gene data so as to obtain a genotyping result; and
a processor signally connected to the genetic testing device and the non-transitory machine readable medium, wherein the processor comprises a polygenic risk score software and a risk assessment program, and the genotyping result is calculated by the polygenic risk score software so as to obtain a polygenic risk score;
wherein the risk assessment program comprises an analysis model, and the polygenic risk score, the clinical data and the at least one genetic marker data are analyzed by the analysis model so as to obtain a risk analysis result, and the risk analysis result is for assessing an age at which the subject is suffered from a chronic kidney disease.

10. The chronic kidney disease risk assessment system of claim 9, wherein the plurality of target gene data comprise ASCC3 gene data, CDC1 gene data, F12 gene data, FAM47 E gene data, FBXO22 gene data, HCRTR2 gene data, KNG1 gene data, LRP2 gene data, RAI14 gene data, NRG4 gene data, PAX8 gene data, PDILT gene data, SIM1 gene data, STC1 gene data, TINAG gene data, UBE2 Q2 gene data and WDR72 gene data.

11. The chronic kidney disease risk assessment system of claim 9, wherein the nucleic acid sample is a blood, a urine, a saliva, or a combination thereof.

12. The chronic kidney disease risk assessment system of claim 9, wherein the plurality of single nucleotide polymorphisms are simultaneously detected by a polymerase chain reaction method, a gene chip analyzing method or a next generation sequencing method.

13. The chronic kidney disease risk assessment system of claim 9, wherein the polygenic risk score software is a PRSice-2 software.

14. The chronic kidney disease risk assessment system of claim 9, wherein the analysis model is obtained by training the plurality of reference polygenic risk score data, the plurality of reference clinical data and the plurality of reference genetic marker data to achieve a convergence by a machine learning algorithm.

15. The chronic kidney disease risk assessment system of claim 14, wherein the machine learning algorithm is a linear regression algorithm, a logistic regression algorithm, a decision tree algorithm or a random forest algorithm.

16. The chronic kidney disease risk assessment system of claim 9, wherein the clinical data comprises an age data, a sex data, a family medical history data, a personal medical history data, a medication history data and a biochemical testing data.

17. The chronic kidney disease risk assessment system of claim 16, wherein the biochemical testing data comprises an estimated glomerular filtration rate data, an albumin concentration data, a calcium concentration data, a phosphate concentration data and a bicarbonate concentration data.
